(19) United States
(12) Patent Application Publication
(10) Pub. No.: US 2025/0127825 A1
(43) Pub. Date: Apr. 24, 2025

(54) PROBIOTIC COMPOSITIONS AND METHODS OF USE THEREOF FOR WEIGHT LOSS

(71) Applicant: SchismBioworks LLC, Broomfield, CO (US)

(72) Inventors: Sebastian Balcombe, Evergreen, CO (US); Martin Purpura, Spring, TX (US); Ralf Jäger, Whitefish Bay, WI (US)

(73) Assignee: Schism Bioworks LLC, Broomfield, CO (US)

(21) Appl. No.: 18/921,986

(22) Filed: Oct. 21, 2024

Related U.S. Application Data
(60) Provisional application No. 63/591,993, filed on Oct. 20, 2023.

(51) Int. Cl. A61K 35/741; A61P 3/04

(52) U.S. Cl. CPC A61K 35/741; A61P 3/04

(57) ABSTRACT

Disclosed herein are compositions for reducing body weight, reducing body fat, and/or preventing obesity and related conditions. In certain aspects, the disclosed methods for reducing body weight, reducing body fat, and/or preventing obesity and related conditions involve administration to subject in need thereof a composition comprising: at least one strain of bacteria wherein the at least one strain of bacteria comprises Propionibacterium freudenreichii UF (UF1). In certain embodiments, the UF1 is probiotic. Further disclosed herein are compositions for the treatment of conditions at least one of obesity, immune system function associated disorders, insulin secretion associated disorders, diabetes, virulence associated conditions, cardiovascular disorder, cardiac disorders, degenerative diseases, sarcopenia, ocular disease, fibrotic diseases, aging associated disorders, improving skin hydration and collagen synthesis, liver disease, Crohn's disease.

### CROSS-REFERENCE TO RELATED APPLICATION(S)

This application claims priority to U.S. Provisional Application No. 63/591,993, filed Oct. 20, 2023, and entitled “PROBIOTIC COMPOSITIONS AND METHODS OF USE THEREOF FOR WEIGHT LOSS”, which is hereby incorporated by reference in its entirety under 35 U.S.C. § 119 (e).

### BACKGROUND

Obesity is a significant problem in the Western world, with estimates of its prevalence ranging from 30% to 50% of the middle-aged population. The number of overweight (defined as a person with a body mass index (BMI) equal to or greater than 25 kg/m²) and obese (defined as a person with a BMI equal to or greater than 30 kg/m²) Americans has continued to increase since 1960, a trend that is not slowing down. Today, approximately 64.5% of adult Americans (about 199 million) are categorized as being overweight or obese. Obesity is becoming a growing concern as the number of people with obesity continues to increase and more is learned about the negative health effects of obesity. Obesity can lead to type 2 diabetes, heart disease, and some cancers. Each year, obesity causes at least 300,000 deaths in the U.S., and healthcare costs of American adults with obesity amount to more than $147 billion. There is a need in the art for compositions and methods to more effectively achieve weight loss.

### BRIEF DESCRIPTION OF THE DRAWINGS

FIG. 1 shows fluorescence of worms after staining with Nile Red and quantitative fluorescence values for worms of different treatments.

FIG. 2 shows fluorescence intensity in worms after staining with Nile Red after different diet treatments, in trial 1.

FIG. 3 shows fluorescence intensity in worms after staining with Nile Red after different diet treatments, in trial 2.

FIG. 4 shows fluorescence intensity in worms after staining with Nile Red after different diet treatments, in trial 3.

FIG. 5 shows fluorescence intensity in worms after staining with Nile Red after different diet treatments, normalized for all trials.

### BRIEF SUMMARY

Described herein are compositions for reducing body weight, reducing body fat, and/or preventing obesity and related conditions. In certain aspects, the disclosed methods for reducing body weight, reducing body fat, and/or preventing obesity and related conditions involve administration to subject in need thereof a composition comprising: at least one strain of bacteria wherein the at least one strain of bacteria comprises Propionibacterium freudenreichii UF (UF1). In certain embodiments, the UF1 is probiotic. In further embodiments, the composition administered delivers a dose of UF1 to the subject from about 5×10⁸ to about 1×10¹¹ colony forming units (CFU). In yet further embodiments, the dose of UF1 is from about 1×10⁹ to about 2×10¹⁰ CFU. in still further embodiments, the dose of UF1 is from about 5×10⁹ to about 1×10¹⁰ CFU. According to certain embodiments, the composition administered delivers a dose of UF1 to the subject from about 5×10⁸ to about 1×10¹¹ Active Fluorescent Units (AFU). In further embodiments, the dose of UF1 is from about 1×10⁹ to about 2×10¹⁰ AFU. In yet further embodiment, the dose of UF1 is from about 5×10⁹ to about 1×10¹⁰ AFUI.

Further disclosed herein are compositions for the treatment of conditions at least one of obesity, immune system function associated disorders, insulin secretion associated disorders, diabetes, virulence associated conditions, cardiovascular disorder, cardiac disorders, degenerative diseases, sarcopenia, ocular disease, fibrotic diseases, aging associated disorders, improving skin hydration and collagen synthesis, liver disease, Crohn's disease.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

A probiotic, such as the composition disclosed herein, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

As used herein, the term “probiotic composition” can refer to a composition containing at least one species, genus, family, strain, order, or class of probiotic bacteria (e.g., a single isolate or a combination of desired bacteria), and can also include any additional carriers, excipients, and/or therapeutic agents that can be administered to a mammal. In certain embodiments, the probiotic composition comprises a buffering agent to allow the probiotic bacteria to survive in the acidic environment of the stomach, that is, the probiotic bacteria resist low pH and are able to survive passage through the stomach to colonize and grow in the intestinal milieu. Buffering agents can include, for example, sodium bicarbonate, milk, yoghurt, infant formula, and other dairy products. In certain embodiments, the probiotic composition is formulated as a food additive. In certain embodiments, the probiotic composition includes other materials known in the art for inclusion in food additives, such as water or other aqueous solutions, starch, binders, thickeners, colorants, flavorants, odorants, acidulants (e.g., lactic acid or malic acid, among others), vitamins, or minerals, among others.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more body weight disorders prior to the administering step. In some aspects of the disclosed method, the subject has been diagnosed with a need for increasing weight loss prior to the administering step.

The term “overweight” is defined as the condition wherein the individual has a BMI greater than or 25 kg/m² and less than 30 kg/m². The terms “overweight” and “pre-obese” are used interchangeably.

As used herein, the term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

As used herein, the terms “manage,” “managing,” and “management” encompass preventing, delaying, or reducing the severity of a recurrence of an adipose associated body composition or body weight disorder, such as obesity, lipodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lipodystrophy, diabetes, metabolic syndrome, fibrosis, cancer and/or changing how a patient responds to the adipose associated body composition or body weight disorder.

As used herein, the term “treatment” refers to the medical management of a patient with the intent to cure, ameliorate, stabilize, or prevent a disease, pathological condition, or disorder. This term includes active treatment, that is, treatment directed specifically to ward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with obesity” means having been subjected to a physical examination by a person of skill, for example, a physician, and found to have a condition that can be diagnosed or treated by a compound or composition that can reduce body mass. As a further example, “diagnosed with a need for weight loss” refers to having been subjected to a physical examination by a person of skill, for example, a physician, and found to have a condition characterized by excess of body fat or other disease wherein decreasing body fat would be beneficial to the subject. Such a diagnosis can be in reference to a disorder, such as obesity, metabolic syndrome, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to excess adipose tissue) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

Disclosed herein are compositions and methods for promoting weight loss and/or preventing obesity. In certain aspects, the disclosed method comprises administering a composition to a subject where the composition comprises Propionibacterium freudenreichii.

Propionibacterium freudenreichii is a gram-positive, non-motile bacterium that plays an important role in the creation of Emmental cheese, and to some extent, Jarlsberg cheese, Leerdammer and Maasdam cheese. Its concentration in Swiss-type cheeses is higher than in any other cheese. Propionibacterium strain, P. UF1, has been isolated from the gut microbiota of preterm infants. P. UF1 is also referred to herein as Propionibacterium freudenreichii UF1 or UF1.

Disclosed herein is a method for increasing vitamin B12 levels in a subject comprising administering to the subject a composition comprising: at least one strain of bacteria wherein the at least one strain of bacteria comprises Propionibacterium freudenreichii UF (UF1). In certain embodiments, the composition administered delivers a dose of UF1 to the subject from about 5×10⁸ to about 1×10¹¹ colony forming units (CFU). In further embodiments, the dose of UF1 provided is from about 1×10⁹ to about 2×10¹⁰ CFU. In still further embodiments, the dose of UF1 is from about 5×10⁹ to about 1×10¹⁰ CFU.

In certain embodiments, at least one strain of bacteria is a probiotic.

In yet further embodiments, the composition further comprises a prebiotic and/or a synbiotic. In even further embodiments, the at least one strain of bacteria is a mixture of a probiotic, prebiotic, and/or synbiotic.

In certain aspects, disclosed herein are methods to promote weight loss through the administration of an effective amount of one or more compositions disclosed herein. According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a patient in need thereof.

Another aspect encompasses a combination therapy to regulate fat storage, energy utilization, and/or weight loss in a subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition comprising UF1 can be administered to the subject prior to, concurrently with or after a gastric bypass or other gastrointestinal or bariatric procedure.

In certain aspects, administration of the disclosed compositions is effective at reducing body weight and/or body fat; preventing an increase in body weight and/or body fat; facilitating a reduction in body weight and/or body fat; preventing, slowing, delaying or treating diseases or conditions attributed to an abnormal accumulation of liver fat; or maintaining and/or improving the insulin sensitivity and/or for treating or preventing hyperinsulinemia and/or insulin resistance.

In certain embodiments, the composition administered delivers a dose of UF1 to the subject from about 5×10⁸ to about 1×10¹¹ colony forming units (CFU). In further embodiments, the dose of UF1 provided is from about 1×10⁹ to about 2×10¹⁰ CFU. In still further embodiments, the dose of UF1 is from about 5×10⁹ to about 1×10¹⁰ CFU.

In certain further embodiments, the amount of UF1 present in a dose is determined by flow cytometry to determine the number of live cells, as measured by Active Fluorescent Units (AFU). In certain aspects, the composition administered delivers a dose of UF1 to the subject from about 5×10⁸ to about 1×10¹¹ AFU. In further embodiments, the dose of UF1 provided is from about 1×10⁹ to about 2×10¹⁰ AFU. In still further embodiments, the dose of UF1 is from about 5×10⁹ to about 1×10¹⁰ AFU.

According to certain embodiments, fat loss is promoted through inducing thermogenesis in the subject. According to exemplary implementations of these embodiments, the composition may also include one or more compounds selected from: caffeine, green tea, capsaicin, garcinia cambogia, yohimbine, and bitter orange.

According to still further embodiments, fat loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides, and octacosanol.

According to certain implementations, the disclosed method further comprises restricting calorie intake of the subject. In exemplary implementations, the amount of fat loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

According to certain embodiments of the disclosed method, the composition is administered in a therapeutically effective amount. In further embodiments, the composition is administered in a prophylactically effective amount.

In some embodiments, contemplated methods may further comprise assessing one or more indices of on-going weight loss, e.g. the ketone body production level in a patient; and optionally adjusting the amount administered; thereby optimizing the therapeutic efficacy of the UF1 composition.

According to certain embodiments, administration of the disclosed compositions can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. For example, when about loss of about 20% body weight, about 30% body weight or more has been achieved. A treatment regimen can include a corrective phase, during which a composition dose sufficient to provide reduction of excess adiposity is administered, followed by a maintenance phase, during which a lower UF1 composition dose sufficient to prevent re-development of excess adiposity is administered.

The disclosed probiotic bacteria according to the invention may be produced using any standard fermentation process known in the art. For example, solid substrate or submerged liquid fermentation. The fermented cultures can be mixed cultures, or single isolates. The probiotic bacteria are anaerobically fermented. The probiotic bacteria are a combination of solid substrate and a submerged liquid fermentation.

The disclosed compositions are formulated for oral administration including chewable foods, beverages, liquids, tablets, capsules, powders, and granulates. In a preferred embodiment the compositions have been formulated into a tablet. In another preferred embodiment the compositions have been formulated into a capsule. In yet another preferred embodiment the compositions have been formulated into granulated or water-soluble powders. Further, in certain embodiments, compositions can be formulated into liquids, creams, lotions, gels dispersions, or ointments for topical administration.

When formulated the composition may contain further ingredients, including ingredients that have a favorable impact on health, flavor, formulating or tableting. Non-limiting examples of additional ingredients that may suitably be incorporated in the present composition are: vitamins, minerals, nutritional supplements (e.g., fiber), fungal extracts, botanical extracts, sweeteners, flow aids, and fillers.

Tableting aids include for example, carboxylic acids such as malic, maleic, citric, iso-citric and succinic, and salts thereof, SiO₂, Aloe Vera, saturated and unsaturated linear and branched fatty acids and their salts, or fatty alcohols. Preferred tableting aides are malic acid, citric acid, stearic acid or Magnesium stearate. Tableting aides are formulated at a concentration of about 1% to 10%, w/w, 2.5% and 7.5 w/w % or any specific value within said range.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension, or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:

1. The various embodiments of clause 1 are directed to a method for reducing body weight in a subject comprising administering to the subject a composition comprising: at least one strain of bacteria wherein the at least one strain of bacteria comprises Propionibacterium freudenreichi UF (UF1).

2. The various embodiments of clause 2 are directed to the method of clause 1, wherein the UF1 is probiotic.

3. The various embodiments of clause 3 are directed to the method of claim 1 or 2, wherein the composition administered delivers a dose of UF1 to the subject from about 5×10⁸ to about 1×10¹¹ colony forming units (CFU).

4. The various embodiments of clause 4 are directed to the method of claim 1, 2, or 3, wherein the dose of UF1 is from about 1×10⁹ to about 2×10¹⁰ CFU.

5. The various embodiments of clause 5 are directed to the method of clause 1, 2, 3, or 4, wherein the dose of UF1 is from about 5×10⁹ to about 1×10¹⁰ CFU.

6. The various embodiments of clause 6 are directed to the method of clause 1, wherein the composition administered delivers a dose of UF1 to the subject from about 5×10⁸ to about 1×10¹¹ Active Fluorescent Units (AFU).

7. The various embodiments of clause 7 are directed to the method of clause 1 or 6, wherein the dose of UF1 is from about 1×10⁹ to about 2×10¹⁰ AFU.

8. The various embodiments of clause 8 are directed to the method of clause 1, 6, or 7, wherein the dose of UF1 is from about 5×10⁹ to about 1×10¹⁰ AFU.

9. The various embodiments of clause 9 are directed to the method of clause 1, wherein the UF1 is probiotic.

10. The various embodiments of clause 10 are directed to the method of clause 1, wherein the UF1 is a probiotic, prebiotic, synbiotic, or mixture of the foregoing.

11. The various embodiments of clause 11 are directed to the method of clause 1, wherein the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii; Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

12. The various embodiments of clause 12 are directed to a method of managing a body weight disorder in a subject in need thereof comprising administering to the subject a therapeutically effective amount of a composition comprising: at least one strain of bacteria wherein the at least one strain of bacteria comprises Propionibacterium freudenreichii UF (UF1).

13. The various embodiments of clause 13 are directed to the method of clause 12, wherein the UF1 is probiotic.

14. The various embodiments of clause 14 are directed to the method of clause 12 or 13, wherein the composition administered delivers a dose of UF1 to the subject from about 5×10⁸ to about 1×10¹¹ colony forming units (CFU).

15. The various embodiments of clause 15 are directed to the method of clause 12, 13, or 14, wherein the dose of UF1 is from about 1×10⁹ to about 2×10¹⁰ CFU.

16. The various embodiments of clause 16 are directed to the method of clause 12, 13, 14, or 15, wherein the dose of UF1 is from about 5×10⁹ to about 1×10¹⁰ CFU.

17. The various embodiments of clause 17 are directed to the method of clause 12 or 13, wherein the composition administered delivers a dose of UF1 to the subject from about 5×10⁸ to about 1×10¹¹ Active Fluorescent Units (AFU).

18. The various embodiments of clause 18 are directed to the method of clause 12, 13 or 17, wherein the dose of UF1 is from about 1×10⁹ to about 2×10¹⁰ AFU.

19. The various embodiments of clause 19 are directed to the method of clause 12, 13, 14, 15, 16, 17, or 18, wherein the body weight disorder is one or more of obesity, lipodystrophy, diabetes or metabolic syndrome, fibrosis and cancer.

20. The various embodiments of clause 20 are directed to the method of clause 12, 13, 14, 15, 16, 17, 18, or 19, wherein the composition is administered in conjunction with calorie restriction of the subject.

21. The various embodiments of clause 21 are directed to the method of clause 12, 13, 14, 15, 16, 17, 18, 19, or 20, wherein the combined administration of the composition and calorie restriction results in greater weight loss in the subject than the weight loss produced by calorie restriction alone.

22. The various embodiments of clause 22 are directed to a method of enhancing endurance in a subject comprising administering to the subject a composition comprising UF1.

23. The various embodiments of clause 23 are directed to the method of clause 22, wherein the composition administered delivers a dose of UF1 to the subject from about 5×10⁸ to about 1×10¹¹ colony forming units (CFU).

24. The various embodiments of clause 24 are directed to the method of clause 22 or 23, wherein the dose of UF1 is from about 1×10⁹ to about 2×10¹⁰ CFU.

25. The various embodiments of clause 25 are directed to the method of clause 22, 23, or 24, wherein the dose of UF1 is from about 5×10⁹ to about 1×10¹⁰ CFU.

26. The various embodiments of clause 26 are directed to the method of clause 22, wherein the composition administered delivers a dose of UF1 to the subject from about 5×10⁸ to about 1×10¹¹ Active Fluorescent Units (AFU).

27. The various embodiments of clause 27 are directed to the method of any preceding clause, wherein the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii; Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

28. The various embodiments of clause 28 are directed to the method of any preceding clause, wherein fat loss is achieved through enhancing lipolysis in the subject.

### EXAMPLES

The following examples are put forth so as to provide those of ordinary skill in the art with a complete disclosure and description of certain examples of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

In this example the weight loss potential of the UF1 probiotic strain was studied. The UF1 probiotic strain was tested in a non-obesogenic setting by measuring the amount of internal fat in UF1-treated worms compared to no treatment controls. Orlistat treatment was also included as a positive control for fat reduction.

Methodology—Worms were raised to the L4 larval stage using standard plate culture methods. The L4 larvae were then loaded into a microfluidic chip and fed a diet of live E. coli OP50 (20 mg/ml) in liquid nematode growth media (NGM). After 24 hours on the chip, the media for each experimental cohort was switched to the experimental diet. The animals were maintained on each experimental diet for 48 hours before being fixed and stained with Nile Red—a fluorescent dye that binds lipids. A fluorescent microscopy system was then used to image the brightness of the lipid staining in the intestine of the worms, which correlates to the levels of fat in each animal.

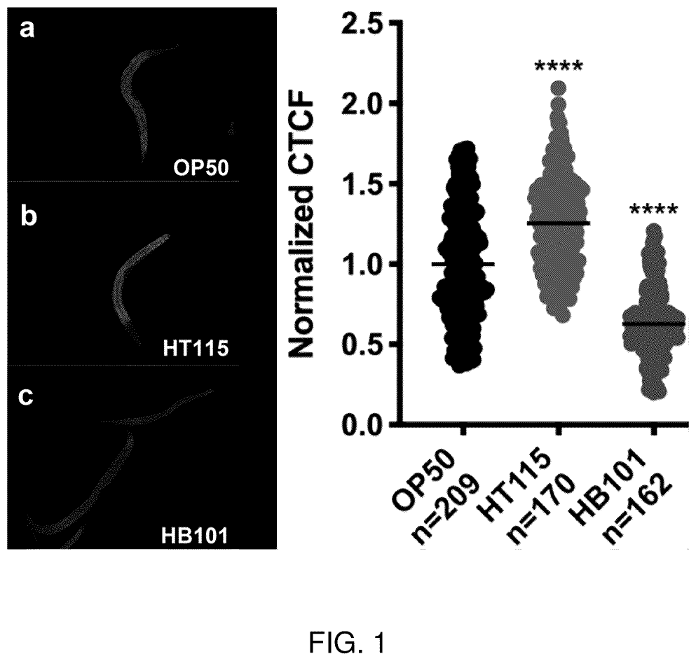

Nile Red gives off fluorescence when illuminated with green light. The brightness of the intestinal saining in each worm per trial was quantified. See FIG. 1. This allows the measurement of the natural variability in fat levels across individuals. ANOVA was then used to determine if the distributions across treatment conditions were statistically different to one another.

TABLE 1

| | Liquid | | |
|---|---|---|---|
| Group | Media | Treatment | Rationale |
| 1 | Standard | Live OP50 20 mg/ml | Probiotic No Treatment |
| | NGM | | Control |
| 2 | Standard | Live OP50 20 mg/ml + | Probiotic Positive Fat |
| | NGM | Orlistat 6 μg/ml | Loss Control |
| 3 | Standard | Probiotic UF1 100 | Probiotic Treatment |
| | NGM | mg/ml* | |
| 4 | Standard | Probiotic UF1 OD 7** | Probiotic Treatment |
| | NGM | | |

*The 100 mg/ml dose of UF1 has an equivalent CFU/ml value to the 20 mg/ml E. coli diet
**An additional UF1 diet was run that had an equivalent OD600 measurement to the 20 mg/ml E. coli diet

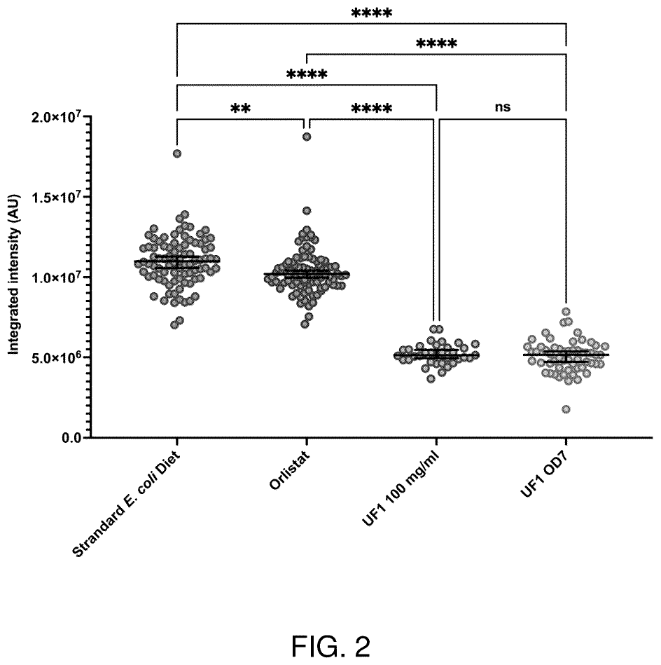

Results—Trial 1—In the first trial of the probiotics in non-obesogenic conditions, the UF1 diets reduced fat levels compared to the standard E. coli OP50 control diet by approximately 53% and 54% respectively. See FIG. 2 and Tables 2-4. The subjects also had significantly less fat than the Orlistat control, with both UF1 diets having approximately 50% and 51% less fat than the Orlistat control.

TABLE 2

| | Standard | | | |
|---|---|---|---|---|
| | E. coli | | UF1 100 | |
| | Diet | Orlistat | mg/ml | UF1 OD7 |
| Number of | 86 | 90 | 35 | 56 |
| values | | | | |
| Minimum | 7014067 | 7054289 | 3663471 | 1763878 |
| 25% Percentile | 10008455 | 9531785 | 4830618 | 4416831 |
| Median | 10985816 | 10198510 | 5124444 | 5147920 |
| 75% Percentile | 12026825 | 10929758 | 5586154 | 5628591 |
| Maximum | 17686716 | 18728496 | 6742484 | 7833343 |
| Range | 10672649 | 11674207 | 3079013 | 6069465 |
| Mean | 10983089 | 10354899 | 5194112 | 5061224 |
| Std. Deviation | 1600306 | 1500279 | 663050 | 1021365 |
| Std. Error of | 172565 | 158143 | 112076 | 136486 |
| Mean | | | | |
| Percentage | 0 | −5.72 | −52.71 | −53.92 |
| Change from | | | | |
| Standard Diet | | | | |
| Percentage | 6.07 | 0 | −49.84 | −51.12 |
| Change from | | | | |
| Orlistat | | | | |

TABLE 3

| | | Below | | | |
|---|---|---|---|---|---|
| Holm-Šidák's multiple | Mean | thresh- | Sum- | Adjusted | |
| comparisons test | Diff. | old? | mary | P Value | |
| Standard E. coli Diet vs. | 628190 | Yes | ** | 0.005 | A-B |
| Orlistat | | | | | |
| Standard E. coli Diet vs. | 5788977 | Yes | **** | <0.0001 | A-C |
| UF1 100 mg/ml | | | | | |
| Standard E. coli Diet vs. | 5921865 | Yes | **** | <0.0001 | A-D |
| UF1 OD7 | | | | | |
| Orlistat vs. UF1 100 | 5160787 | Yes | **** | <0.0001 | B-C |
| mg/ml | | | | | |
| Orlistat vs. UF1 OD7 | 5293675 | Yes | **** | <0.0001 | B-D |
| UF1 100 mg/ml vs. UF1 | 132888 | No | ns | 0.6519 | C-D |
| OD7 | | | | | |

TABLE 4

| | | | Mean | SE of | | | | |
|---|---|---|---|---|---|---|---|---|
| Test details | Mean 1 | Mean 2 | Diff. | diff. | n1 | n2 | t | DF |
| Standard E. coli | 10983089 | 10354899 | 628190 | 205898 | 86 | 90 | 3.051 | 263 |
| Diet vs. Orlistat | | | | | | | | |
| Standard E. coli | 10983089 | 5194112 | 5788977 | 273764 | 86 | 35 | 21.15 | 263 |
| Diet vs. UF1 100 | | | | | | | | |
| mg/ml | | | | | | | | |
| Standard E. coli | 10983089 | 5061224 | 5921865 | 234459 | 86 | 56 | 25.26 | 263 |
| Diet vs. UF1 OD7 | | | | | | | | |
| Orlistat vs. UF1 | 10354899 | 5194112 | 5160787 | 271998 | 90 | 35 | 18.97 | 263 |
| 100 mg/ml | | | | | | | | |
| Orlistat vs. UF1 | 10354899 | 5061224 | 5293675 | 232396 | 90 | 56 | 22.78 | 263 |
| OD7 | | | | | | | | |
| UF1 100 mg/ml | 5194112 | 5061224 | 132888 | 294211 | 35 | 56 | 0.4517 | 263 |
| vs. UF1 OD7 | | | | | | | | |

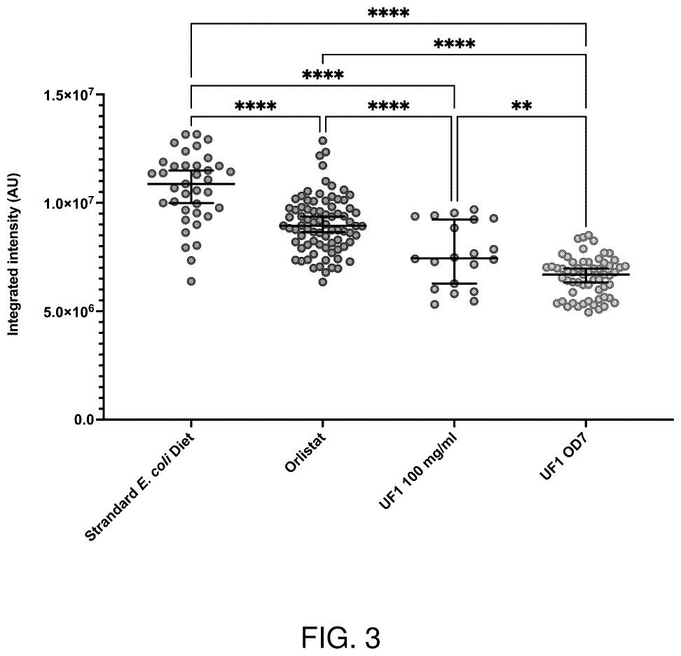

Trial 2—In the second trial of the probiotics in non-obesogenic conditions, the UF1 diets reduced fat levels compared to the standard E. coli OP50 control diet by approximately 28% and 38%, respectively. The subjects also had significantly less fat than the Orlistat control, with both UF1 diets having approximately 16% and 27% less fat than the Orlistat control. See FIG. 3 and Tables 5-7.

TABLE 5

| | Standard | | | |
|---|---|---|---|---|
| | E. coli | | UF1 100 | |
| | Diet | Orlistat | mg/ml | UF1 OD7 |
| Number of | 37 | 79 | 20 | 59 |
| values | | | | |
| Minimum | 6374505 | 6339621 | 5308005 | 4944872 |
| 25% Percentile | 9590671 | 8068243 | 6071943 | 5641382 |
| Median | 10870079 | 8938493 | 7441676 | 6691779 |
| 75% Percentile | 11706140 | 9795496 | 9266412 | 7221991 |
| Maximum | 13154246 | 12863469 | 9683312 | 8497751 |
| Range | 6779741 | 6523848 | 4375307 | 3552879 |
| Mean | 10633818 | 9011934 | 7612410 | 6576861 |
| Std. Deviation | 1652198 | 1316062 | 1490478 | 923188 |
| Std. Error of | 271620 | 148068 | 333281 | 120189 |
| Mean | | | | |
| Percentage | 0 | −15.25 | −28.41 | −38.15 |
| Change from | | | | |
| Standard Diet | | | | |
| Percentage | 18.00 | 0 | −15.53 | −27.02 |
| Change from | | | | |
| Orlistat | | | | |

TABLE 6

| | | Below | | | |
|---|---|---|---|---|---|
| Holm-Šidák's multiple | Mean | thresh- | Sum- | Adjusted | |
| comparisons test | Diff. | old? | mary | P Value | |
| Standard E. coli Diet vs. | 1621884 | Yes | **** | <0.0001 | A-B |
| Orlistat | | | | | |
| Standard E. coli Diet vs. | 3021408 | Yes | **** | <0.0001 | A-C |
| UF1 100 mg/ml | | | | | |
| Standard E. coli Diet vs. | 4056957 | Yes | **** | <0.0001 | A-D |
| UF1 OD7 | | | | | |
| Orlistat vs. UF1 100 | 1399524 | Yes | **** | <0.0001 | B-C |
| mg/ml | | | | | |
| Orlistat vs. UF1 OD7 | 2435073 | Yes | **** | <0.0001 | B-D |
| UF1 100 mg/ml vs. UF1 | 1035549 | Yes | ** | 0.0025 | C-D |
| OD7 | | | | | |

TABLE 7

| | | | Mean | SE of | | | | |
|---|---|---|---|---|---|---|---|---|
| Test details | Mean 1 | Mean 2 | Diff. | diff. | n1 | n2 | t | DF |
| Standard E. coli Diet | 10633818 | 9011934 | 1621884 | 259864 | 37 | 79 | 6.241 | 191 |
| vs. Orlistat | | | | | | | | |
| Standard E. coli Diet | 10633818 | 7612410 | 3021408 | 362037 | 37 | 20 | 8.346 | 191 |
| vs. UF1 100 mg/ml | | | | | | | | |
| Standard E. coli Diet | 10633818 | 6576861 | 4056957 | 273552 | 37 | 59 | 14.83 | 191 |
| vs. UF1 OD7 | | | | | | | | |
| Orlistat vs. UF1 100 | 9011934 | 7612410 | 1399524 | 326528 | 79 | 20 | 4.286 | 191 |
| mg/ml | | | | | | | | |
| Orlistat vs. UF1 | 9011934 | 6576861 | 2435073 | 224456 | 79 | 59 | 10.85 | 191 |
| OD7 | | | | | | | | |
| UF1 100 mg/ml vs. | 7612410 | 6576861 | 1035549 | 337524 | 20 | 59 | 3.068 | 191 |
| UF1 OD7 | | | | | | | | |

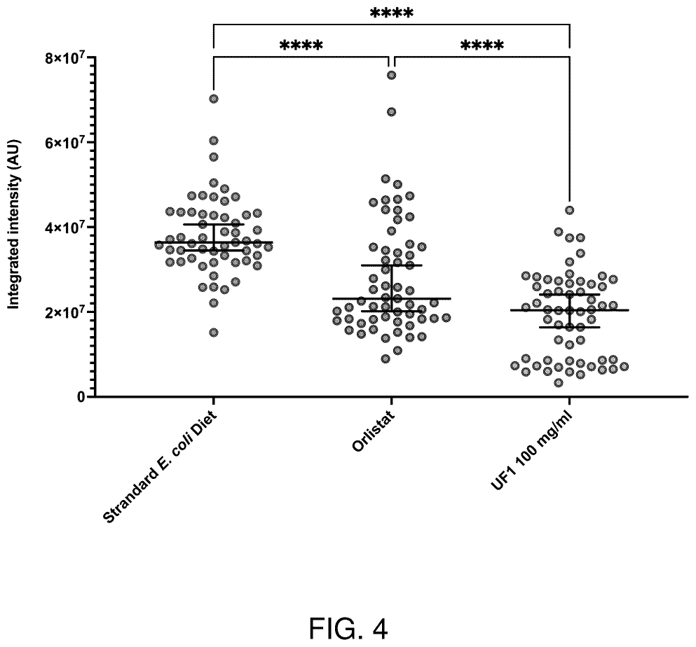

Trial 3—In the third trial of the probiotics in non-obesogenic conditions, the UF1 diets reduced fat levels compared to the standard E. coli OP50 control diet by approximately 50%. The subjects also had significantly less fat than the Orlistat control, with both UF1 100 mg/ml diets having approximately 32% less fat than the Orlistat control. See FIG. 4 and Tables 8-10.

TABLE 8

| | Strandard E. coli | | UF1 100 |
|---|---|---|---|
| | Diet | Orlistat | mg/ml |
| Number of values | 55 | 59 | 58 |
| Minimum | 15140966 | 8941802 | 3274146 |
| 25% Percentile | 32034464 | 18305020 | 8534585 |
| Median | 36389764 | 23127648 | 20425415 |
| 75% Percentile | 43236784 | 35318592 | 26800917 |
| Maximum | 70203760 | 75769432 | 43942216 |
| Range | 55062794 | 66827630 | 40668070 |
| Mean | 38001780 | 28212711 | 19167276 |
| Std. Deviation | 9326914 | 13777764 | 10147773 |
| Std. Error of Mean | 1257641 | 1793712 | 1332468 |
| Percentage Change | 0 | −25.76 | −49.56 |
| from Standard Diet | | | |
| Percentage Change | 34.70 | 0 | −32.06 |
| from Orlistat | | | |

TABLE 9

| | | Below | | | |
|---|---|---|---|---|---|
| Holm-Šidák's multiple | Mean | thresh- | Sum- | Adjusted | |
| comparisons test | Diff. | old? | mary | P Value | |
| Standard E. coli Diet vs. | 9789069 | Yes | **** | <0.0001 | A-B |
| Orlistat | | | | | |
| Standard E. coli Diet vs. | 18834504 | Yes | **** | <0.0001 | A-C |
| UF1 100 mg/ml | | | | | |
| Orlistat vs. UF1 100 | 9045435 | Yes | **** | <0.0001 | B-C |
| mg/ml | | | | | |

TABLE 10

| | | | Mean | SE of | | | | |
|---|---|---|---|---|---|---|---|---|
| Test details | Mean 1 | Mean 2 | Diff. | diff. | n1 | n2 | t | DF |
| Standard E. coli | 38001780 | 28212711 | 9789069 | 2117869 | 55 | 59 | 4.622 | 169 |
| Diet vs. Orlistat | | | | | | | | |
| Standard E. coli | 38001780 | 19167276 | 18834504 | 2126660 | 55 | 58 | 8.856 | 169 |
| Diet vs. UF1 100 | | | | | | | | |
| mg/ml | | | | | | | | |
| Orlistat vs. UF1 | 28212711 | 19167276 | 9045435 | 2089330 | 59 | 58 | 4.329 | 169 |
| 100 mg/ml | | | | | | | | |

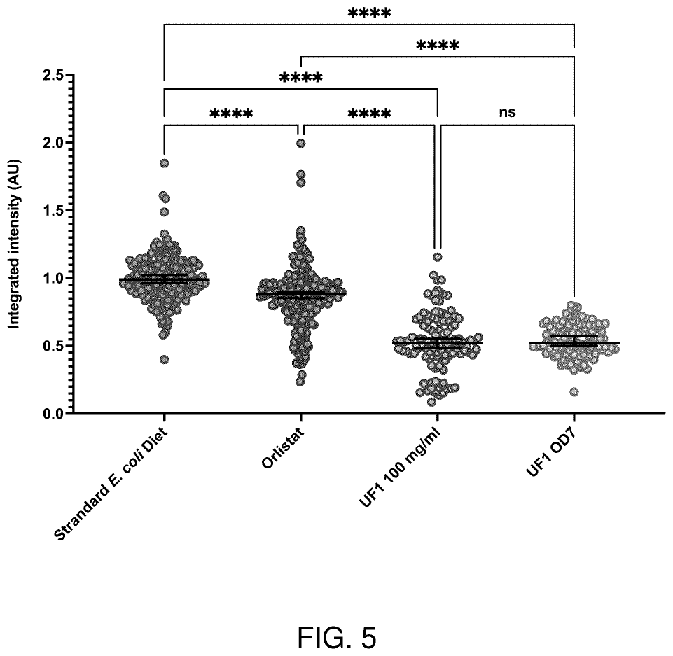

All datasets were combined by normalizing the data in each trial by dividing it by the mean fluorescence value of the standard diet. In the combined data, the UF1 diets reduced fat levels compared to the standard E. coli OP50 control diet by approximately 47% and 46%, respectively. The subjects also had reduced fat levels compared to the Orlistat control by approximately 38% and 37%, respectively. See FIG. 5 and Tables 11-13.

TABLE 11

| | Standard | | | |
|---|---|---|---|---|
| | E. coli | | UF1 100 | |
| | Diet | Orlistat | mg/ml | UF1 OD7 |
| Number of | 178 | 228 | 113 | 115 |
| values | | | | |
| Minimum | 0.3984 | 0.2353 | 0.08616 | 0.1606 |
| 25% Percentile | 0.8983 | 0.7519 | 0.4246 | 0.4683 |
| Median | 0.9903 | 0.8801 | 0.5247 | 0.5207 |
| 75% Percentile | 1.107 | 0.9596 | 0.6888 | 0.644 |
| Maximum | 1.847 | 1.994 | 1.156 | 0.7991 |
| Range | 1.449 | 1.759 | 1.07 | 0.6385 |
| Mean | 1 | 0.8579 | 0.5321 | 0.5417 |
| Std. Deviation | 0.183 | 0.2292 | 0.2197 | 0.1195 |
| Std. Error of | 0.01372 | 0.01518 | 0.02066 | 0.01114 |
| Mean | | | | |
| Percentage | 0 | −14.21 | −46.79 | −45.83 |
| Change from | | | | |
| Standard Diet | | | | |
| Percentage | 16.56 | 0 | −37.98 | −36.86 |
| Change from | | | | |
| Orlistat | | | | |

TABLE 12

| Holm-Šídák's | | | | | | |
|---|---|---|---|---|---|---|
| multiple | Mean | Below | | Adjusted | | |
| comparisons test | Diff. | threshold? | Summary | P Value | | |
| Standard E. coli Diet | 0.1421 | 0.09088 to | Yes | **** | <0.0001 | A-B |
| vs. Orlistat | | 0.1933 | | | | |
| Standard E. coli Diet | 0.4679 | 0.4064 to | Yes | **** | <0.0001 | A-C |
| vs. UF1 100 mg/ml | | 0.5295 | | | | |
| Standard E. coli Diet | 0.4583 | 0.3970 to | Yes | **** | <0.0001 | A-D |
| vs. UF1 OD7 | | 0.5195 | | | | |
| Orlistat vs. UF1 100 | 0.3259 | 0.2670 to | Yes | **** | <0.0001 | B-C |
| mg/ml | | 0.3847 | | | | |
| Orlistat vs. UF1 OD7 | 0.3162 | 0.2577 to | Yes | **** | <0.0001 | B-D |
| | | 0.3748 | | | | |
| UF1 100 mg/ml vs. | −0.009643 | −0.07745 to | No | ns | 0.9832 | C-D |
| UF1 OD7 | | 0.05816 | | | | |

TABLE 13

| | | | Mean | SE of | | | | |
|---|---|---|---|---|---|---|---|---|
| Test details | Mean 1 | Mean 2 | Diff. | diff. | n1 | n2 | t | DF |
| Standard E. coli Diet | 1 | 0.8579 | 0.1421 | 0.01988 | 178 | 228 | 10.11 | 630 |
| vs. Orlistat | | | | | | | | |
| Standard E. coli Diet | 1 | 0.5321 | 0.4679 | 0.0239 | 178 | 113 | 27.68 | 630 |
| vs. UF1 100 mg/ml | | | | | | | | |
| Standard E. coli Diet | 1 | 0.5417 | 0.4583 | 0.02378 | 178 | 115 | 27.26 | 630 |
| vs. UF1 OD7 | | | | | | | | |
| Orlistat vs. UF1 100 | 0.8579 | 0.5321 | 0.3259 | 0.02286 | 228 | 113 | 20.16 | 630 |
| mg/ml | | | | | | | | |
| Orlistat vs. UF1 OD7 | 0.8579 | 0.5417 | 0.3162 | 0.02273 | 228 | 115 | 19.67 | 630 |
| UF1 100 mg/ml vs. | 0.5321 | 0.5417 | −0.009643 | 0.02632 | 113 | 115 | 0.518 | 630 |
| UF1 OD7 | | | | | | | | |

Conclusions—The probiotic UF1 reduced the levels of fat in the intestines of C. elegans worms raised under non-obesogenic conditions. UF1 was examined using two different dietary regimes. The first dietary regime used CFU equivalence to the standard lab diet of 20 mg/ml E. coli. The second dietary regime used OD600 equivalence to the standard lab diet of 20 mg/ml E. coli. The UF1 diets also induced a stronger reduction in fat level than the anti-obesity drug Orlistat.

Although the disclosure has been described with reference to preferred embodiments, persons skilled in the art will recognize that changes may be made in form and detail without departing from the spirit and scope of the disclosed apparatus, systems and methods.

What is claimed is:

1. A method for reducing body weight in a subject comprising administering to the subject a composition comprising: at least one strain of bacteria wherein the at least one strain of bacteria comprises Propionibacterium freudenreichii UF (UF1).

2. The method of claim 1, wherein the UF1 is probiotic.

3. The method of claim 2, wherein the composition administered delivers a dose of UF1 to the subject from about 5×10⁸ to about 1×10¹¹ colony forming units (CFU).

4. The method of claim 3, wherein the dose of UF1 is from about 5×10⁹ to about 1×10¹⁰ CFU.

5. The method of claim 1, wherein the composition administered delivers a dose of UF1 to the subject from about 5×10⁸ to about 1×10¹¹ Active Fluorescent Units (AFU).

6. The method of claim 5, wherein the dose of UF1 is from about 5×10⁹ to about 1×10¹⁰ AFU.

7. The method of claim 1, wherein the UF1 is a probiotic, prebiotic, synbiotic, or mixture of the foregoing.

8. The method of claim 1, wherein the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii; Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

9. A method of managing a body weight disorder in a subject in need thereof comprising administering to the subject a therapeutically effective amount of a composition comprising: at least one strain of bacteria wherein the at least one strain of bacteria comprises Propionibacterium freudenreichii UF (UF1).

10. The method of claim 9, wherein the UF1 is probiotic.

12. The method of claim 10, wherein the composition administered delivers a dose of UF1 to the subject from about 5×10⁸ to about 1×10¹¹ colony forming units (CFU).

13. The method of claim 12, wherein the dose of UF1 is from about 5×10⁹ to about 1×10¹⁰ CFU.

14. The method of claim 11, wherein the dose of UF1 is from about 1×10⁹ to about 2×10¹⁰ AFU.

15. The method of claim 10, wherein the body weight disorder is one or more of obesity, lipodystrophy, diabetes, metabolic syndrome, fibrosis and cancer.

16. The method of claim 15, wherein the composition is administered in conjunction with calorie restriction of the subject.

17. The method of claim 16, wherein the combined administration of the composition and calorie restriction results in greater weight loss in the subject than the weight loss produced by calorie restriction alone.

18. A method of enhancing endurance in a subject comprising administering to the subject a composition comprising UF1.

19. The method of claim 18, wherein the composition administered delivers a dose of UF1 to the subject from about 5×10⁸ to about 1×10¹¹ colony forming units (CFU).

20. The method of claim 19, wherein the dose of UF1 is from about 5×10⁹ to about 1×10¹⁰ CFU.
